(19) United States
(12) Patent Application Publication
(10) Pub. No.: US 2009/0228037 A1
(43) Pub. Date: Sep. 10, 2009

(54) Vascular Closure Implant

(75) Inventor: Richard Rego, Mansfield, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc, Santa Rosa, CA (US)

(21) Appl. No.: 12/044,594

(22) Filed: Mar. 7, 2008

(51) Int. Cl. A61B 17/08

(52) U.S. Cl. 606/213

(57) ABSTRACT

An implant for closing an arteriotomy includes a head having a proximal surface and a distal surface. A tapered plug extends from the distal surface of the head. A plurality of prongs also extends from the distal surface of the head. Upon implantation, the distal surface of the head faces an outside surface of the vessel wall, the tapered plug is disposed within the passage, and the prongs are disposed in tissue of the vessel wall surrounding the passage. The prongs may include a barb to assist in embedding the prongs in the tissue surrounding the puncture. The implant may be bioabsorbable.

### FIELD OF THE INVENTION

The present disclosure relates to a device and method for closing an opening in a vessel wall after a medical procedure, and in particular, to a device and method for closing a puncture arteriotomy after an intra-luminal procedure such as catheterization.

### BACKGROUND OF THE INVENTION

Catheters and catheterization procedures for diagnosis or treatment of cardiovascular and/or peripheral vascular diseases are well known, and typically involve the Seldinger technique to make insertions through layers of tissue and through a wall of the femoral artery. After a diagnostic or interventional catheterization, the arteriotomy puncture formed by the catheter or introducer sheath must be closed. The puncture opening in the artery typically ranges from 5 French (0.0655 inch, 1.67 mm) such as for a diagnostic angiography procedure to as large as 30 French (0.393 inch, 10.00 mm) for an interventional procedure such as implanting an inferior vena cava (IVC) filter. Traditionally, intense pressure has been applied to the puncture site for at least 30-45 minutes after removal of the catheter. Patients who have had a femoral artery puncture are then required to remain at bed rest, essentially motionless and often with a heavy sandbag placed on their upper legs, for several hours to ensure that the bleeding has stopped.

Other proposed methods or devices for sealing vascular punctures include the use of a biodegradable plugs, percutaneous suturing devices, staples and surgical clips, and skin seals. However, there is still a need for a more effective method and device for sealing punctures or other passages through tissue, e.g., an opening into a blood vessel.

### BRIEF SUMMARY OF THE INVENTION

An implant for sealing a passage through a vessel wall is presented. The implant includes a head having a proximal surface and a distal surface. A tapered plug extends from the distal surface of the head. A plurality of prongs also extends from the distal surface of the head. Upon implantation, the distal surface of the head faces an outside surface of the vessel wall, the tapered plug is disposed within the passage, and the prongs are disposed in tissue of the vessel wall surrounding the passage. The prongs may include a barb to assist in embedding the prongs in the tissue surrounding the puncture. The tapered plug is larger near the distal surface of the head and smaller as the prong extends distally away from the head. The tapered plug may include a proximal end that is generally oblong in shape and a distal end that is generally circular in shape. The implant may be bioabsorbable.

### BRIEF DESCRIPTION OF DRAWINGS

The foregoing and other features and advantages of the present disclosure will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 is top perspective view of an implant in accordance with an embodiment of the present invention.

FIG. 2 is bottom perspective view of the implant of FIG. 1.

FIG. 3 is another bottom perspective view of the implant of FIG. 1.

FIG. 4 is a side, partial cut-away view of delivery device in accordance with an embodiment of the present invention.

FIG. 5 is cut-away view of an embodiment of a handle of the delivery device of FIG. 4.

FIG. 6 is a perspective view of an embodiment of a retention mechanism of the delivery device of FIG. 4 in the undeployed position.

FIG. 7 is a perspective view of the retention mechanism of FIG. 6 in the deployed position.

FIG. 8 is perspective view of the retention mechanism of FIG. 6 nested within an embodiment of a forward cam follower of the delivery device of FIG. 4.

FIG. 9 is perspective view of an embodiment of a slide component disposed on the retention mechanism of FIG. 8.

FIG. 10 is side, partial cut-away view of the delivery device of FIG. 4 mated with a sheath.

FIG. 11 is a perspective view of the delivery device of FIG. 4 and the sheath of FIG. 10 delivered to a vessel.

FIG. 12 is a perspective view of an embodiment of retention feet of the delivery device of FIG. 4 with the retention feet deployed inside a vessel.

FIG. 13 is a perspective, partial cut-away view of the delivery device of FIG. 4 with the trigger of the handle rotated.

FIGS. 14 and 15 are perspective views of the implant of FIG. 1 being implanted in an arteriotomy.

FIG. 16 is a perspective, partial cut-away view of the delivery device of FIG. 4 with the trigger rotated more than in FIG. 13.

FIG. 17 is a perspective view of the retention feet being undeployed.

FIG. 18 is a perspective view of the retention feet completely undeployed.

FIG. 19 is a perspective view of the implant of FIG. 1 implanted into an arteriotomy after the delivery device has been withdrawn.

### DETAILED DESCRIPTION OF THE INVENTION

Specific embodiments of the present disclosure are now described with reference to the figures, where like reference numbers indicate identical or functionally similar elements. The present disclosure is directed to a vascular closure implant and a method for closing an arteriotomy using such a vascular closure implant after a medical procedure in which a blood vessel wall was punctured to gain access to the vessel lumen.

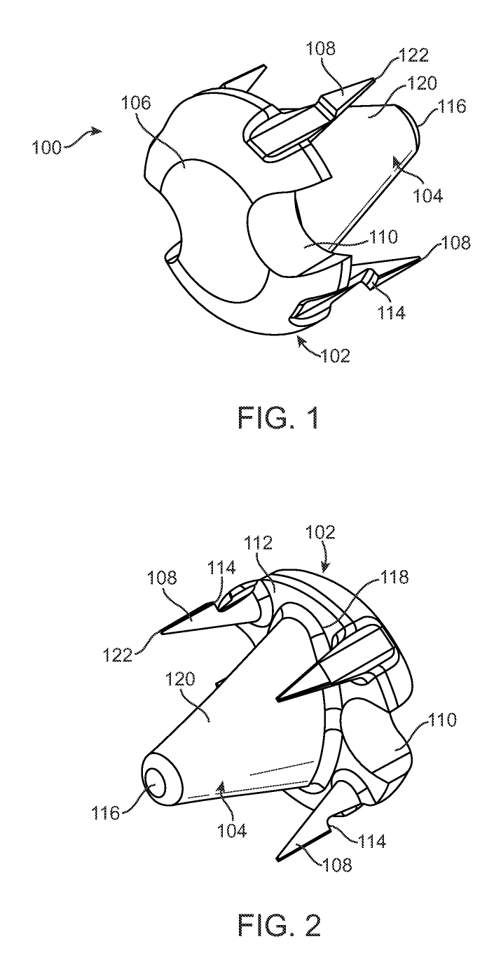

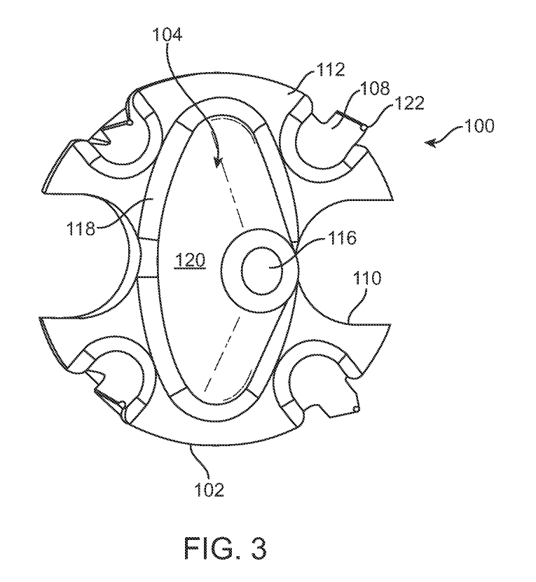

FIGS. 1-3 show perspective views of a vascular closure implant 100 in accordance with an embodiment of the present invention. Implant 100 includes a head 102 and a tapered plug 104. Head 102 is generally shaped like a disc or button and includes a proximally facing surface 106 and an opposite, distally facing surface 112. Tapered plug 104 extends from distally facing surface 112. Tapered plug 104 can be unitary with head 102, or may be a separate piece attached to head 102 by techniques such as insert molding, adhesive bonding, solvent bonding, or melt bonding, e.g. ultrasonic welding, vibration welding or spin welding. Tapered plug 104 includes a proximal end 118 adjoining distally facing surface 112 of head 102, a distal end 116, and a generally conical surface 120 disposed there between. In the embodiment shown in FIGS. 1-3, proximal end 118 is generally oblong or elliptically shaped and distal end 116 is generally circular in shape, with surface 120 being tapered there between. In an alternative embodiment (not shown), plug proximal end 118 is generally circular.

Implant 100 further includes a plurality of prongs 108 extending generally in the distal direction from head 102. In the embodiment shown in FIGS. 1-3, implant 100 includes four prongs 108 spaced equally around head 102. Prongs 108 may be oriented generally orthogonally from head distal surface 112 or splayed to pierce the vessel wall somewhat farther from the arteriotomy and to thereby encompass more arterial tissue. Each prong 108 includes a pointed tip 122 and a barb 114. Barbs 114 aid in grasping tissue of a vessel wall when implanted in a vessel. As will be understood by those of skill in the art, prongs 108 may number two, three, four, or more. Prongs 108 can be unitary with head 102, or may be separate pieces attached to head 102. Prongs 108 can be of the same or different lengths, and may be shorter, longer, or the same length as tapered plug 104. In one embodiment, prongs 108 are short enough to pierce the vessel wall without entering the lumen of the blood vessel where prong tips 122 could be potential sites for thrombus formation.

Head 102 of implant 100 may also include slots 110 on opposite sides thereof. Slots 110 are essentially cut-outs or notches where the continuous circular shape of head 102 is interrupted, but it is understood that slots 110 need not be cut-out of head 102, but instead may be formed in head 102 when head 102 is formed. Slots 102 may be semi-circular in shape and are sized and shaped to allow retention wires (described in more detail below) to pass there through. As best seen in FIG. 3, the long axis of the oblong shaped proximal end 118 of plug 104 extends in a direction generally orthogonal to an axis extending between slots 110. In an alternative embodiment, slots 110 may be aligned with the long axis of the oblong shaped proximal end 118 of plug 104 (not shown).

Implant 100 may be made of biocompatible metallic or polymeric materials. Prongs 108 are made of a rigid material so that they can pierce the tissue surrounding an arteriotomy. Head 102 and plug 104 may be made of either rigid or semi-rigid materials. In embodiments where head 102, tapered plug 104 and/or prongs 108 are made as separate pieces, as described above, the components may be made of the same or dissimilar materials. In one example, implant 100 may comprise unitary head and prongs 102, 108 made of biocompatible metal by techniques such as stamping, casting or metal-injection-molding (MIM) and plug 104 may be made of a biocompatible polymer and attached to head 102 by suitable techniques that may be selected from the methods described above.

Biocompatible metals suitable for use in implant 110 include stainless steel 316L, stainless steel 316 LVM, titanium or bioabsorbable magnesium, which is absorbed by a patient's body as the arteriotomy into which implant 100 is inserted heals. Biocompatible non-resorbable polymeric materials suitable for use in implant 110 may include polymethylmethacrylate (PMMA), high density polyethylene (HDPE), and ultra high molecular weight polyethylene (UHMWPE). Implant 100 is preferably made as a unitary construction of a rigid implant grade bioabsorbable polymer material such that implant 100 is absorbed by a patient's body as the arteriotomy into which implant 100 is inserted heals. For example, and not by way of limitation, implant 100 may be made from polyglycolic acid (PGA), polylactic acid (PLA), alloys or blends of PGA and PLA, alloys or blends of PGA and tri-methyl carbonate, and alloys or blends of PLA and tri-methyl carbonate.

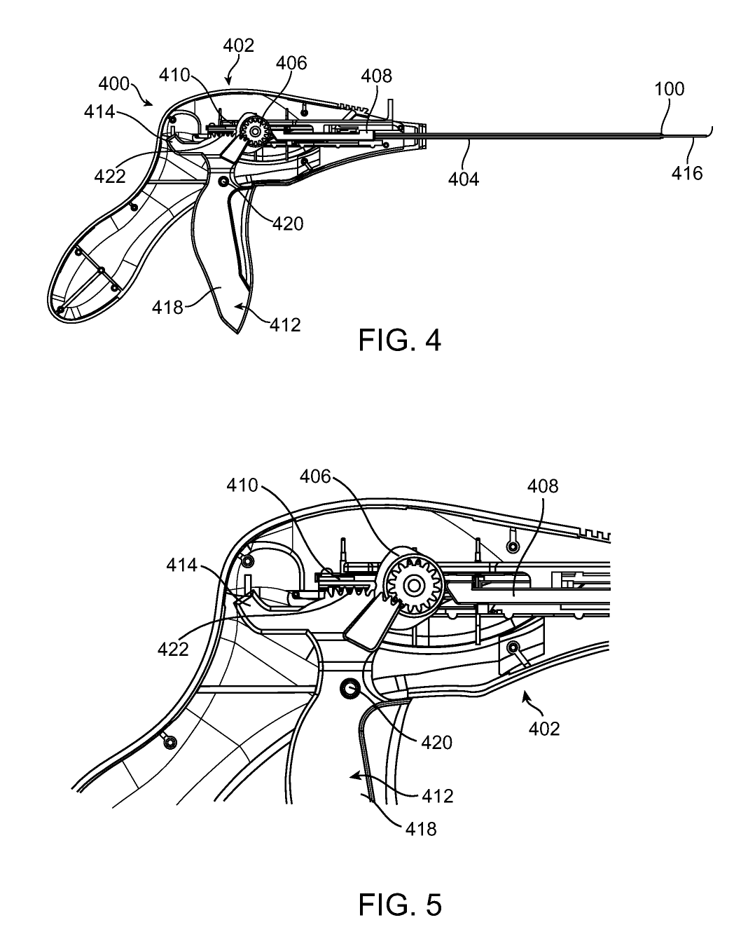

FIGS. 4-9 illustrate an embodiment of a device for delivering and implanting an implant such as implant 100 to an arteriotomy in a vessel following an intra-luminal procedure. In particular, FIG. 4 illustrates a cut-away view of a delivery device 400. Delivery device 400 includes a handle 402, a push rod 404, and retention feet 416. Implant 100 is disposed at a distal end of push rod 404.

Handle 402 is shown in greater detail in FIG. 5. Handle 402 includes a trigger 412, a cam gear 406, a rear cam follower 410, and a front cam follower 408. Trigger 412 includes a handle 418, pivot point 420, and a rack 422. Rack 422 includes teeth that engage teeth of cam gear 406. Trigger 412 further includes a rear arm projection 414. Cam gear 406 is disposed between rear cam follower 410 and front cam follower 408 such that rotation of cam gear 406 translates into linear motion of rear cam follower 410 and/or front cam follower 408.

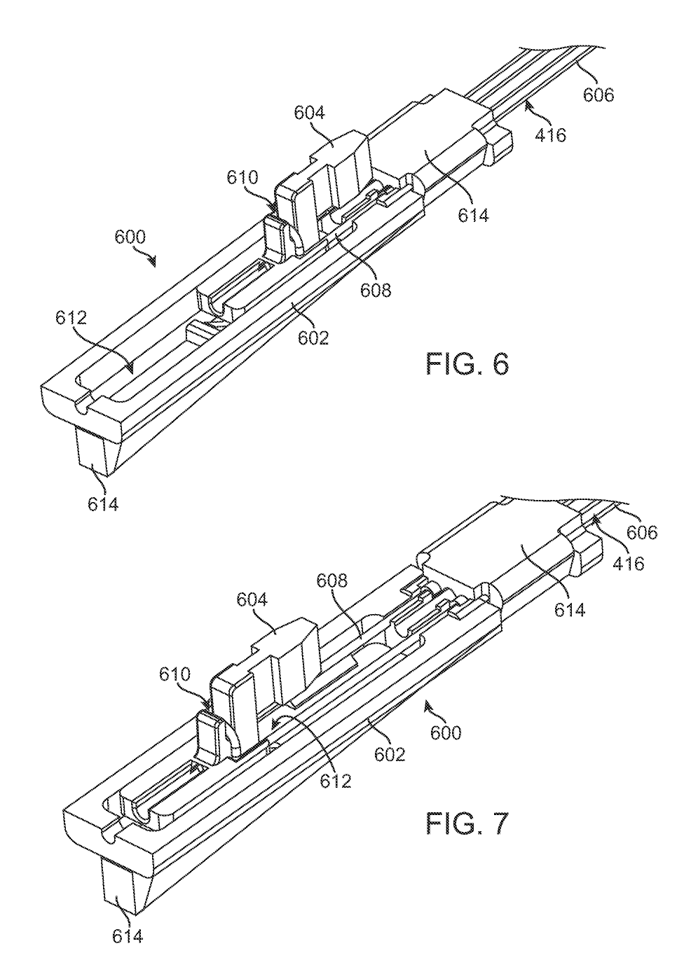

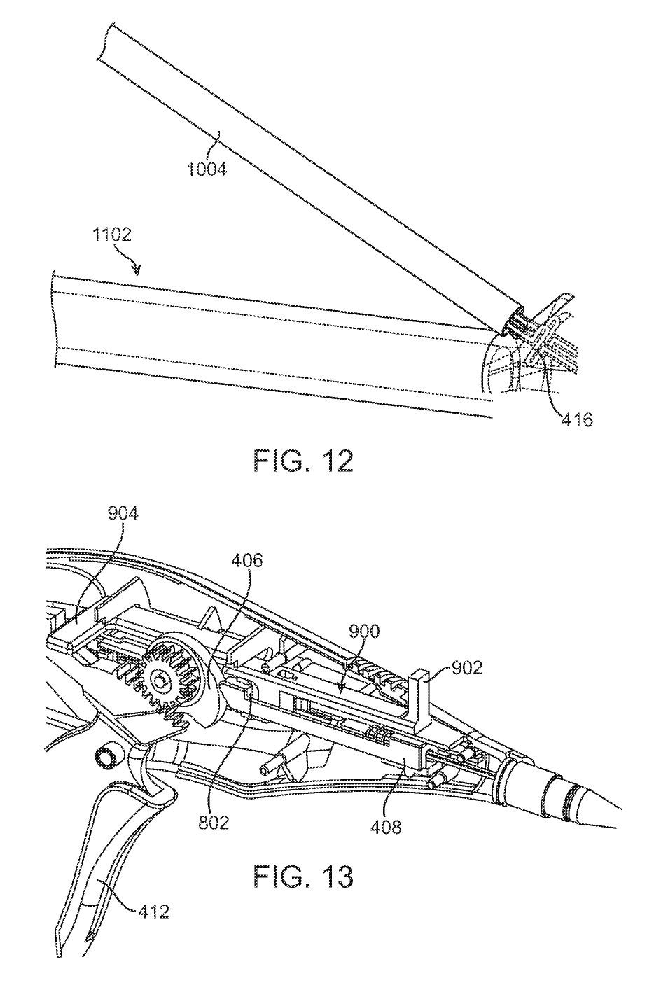

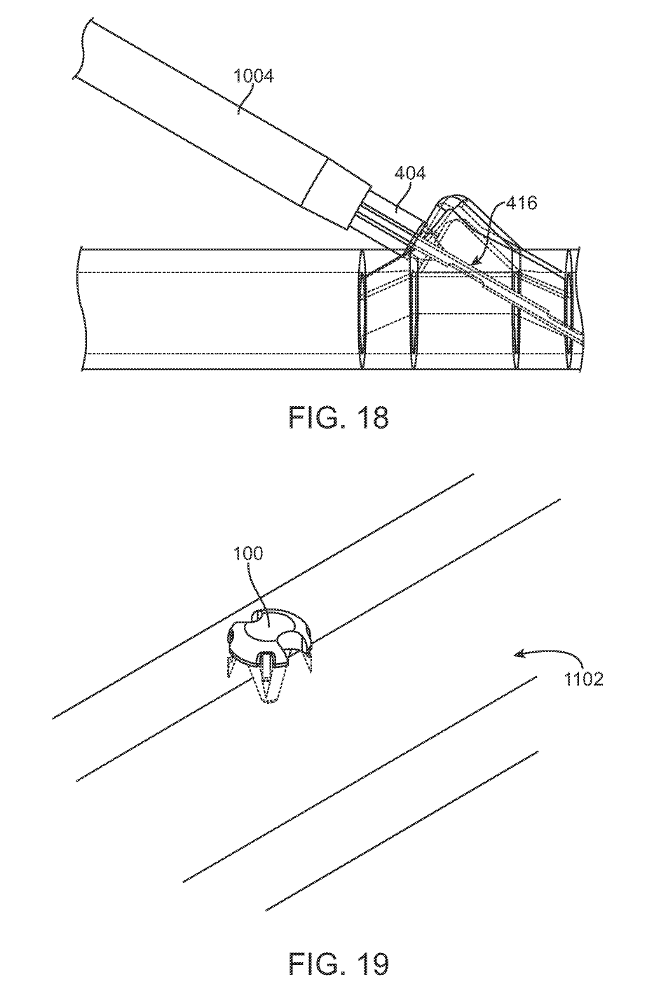

FIGS. 6 and 7 illustrate a retention mechanism 600. Retention mechanism 600 includes a tube holder component 602, a wire holder component 604, and retention feet 416. Retention feet 416 each include a tube 606 and a wire 608 disposed within tube 606. Wire 608 is slidable within tube 606, although a distal portion of wire 608 is coupled to a distal portion of tube 606 to deploy retention feet 416, as described in more detail below. Wire 608 is looped through a slot 610 in wire holder component 604. Wire holder component 604 is moveable within a slot 612 through tube holder component 602. A distal portion 614 of tube holder 608 holds tubes 606. Tube holder component 602 further includes an extension 614 extending downward from a proximal end of tube holder component 602. FIG. 6 illustrates wire holder component 604 in a distal position corresponding to the retention feet 416 in an undeployed position in which retention feet 416 are generally straight, as shown in FIGS. 11 and 18. FIG. 7 illustrates wire holder component 604 in a proximal position corresponding to retention feet 416 in a deployed position in which retention feet 416 include a portion that is disposed generally transverse to the longitudinal axis of retention feet 416, as shown in FIGS. 12, 14, and 15.

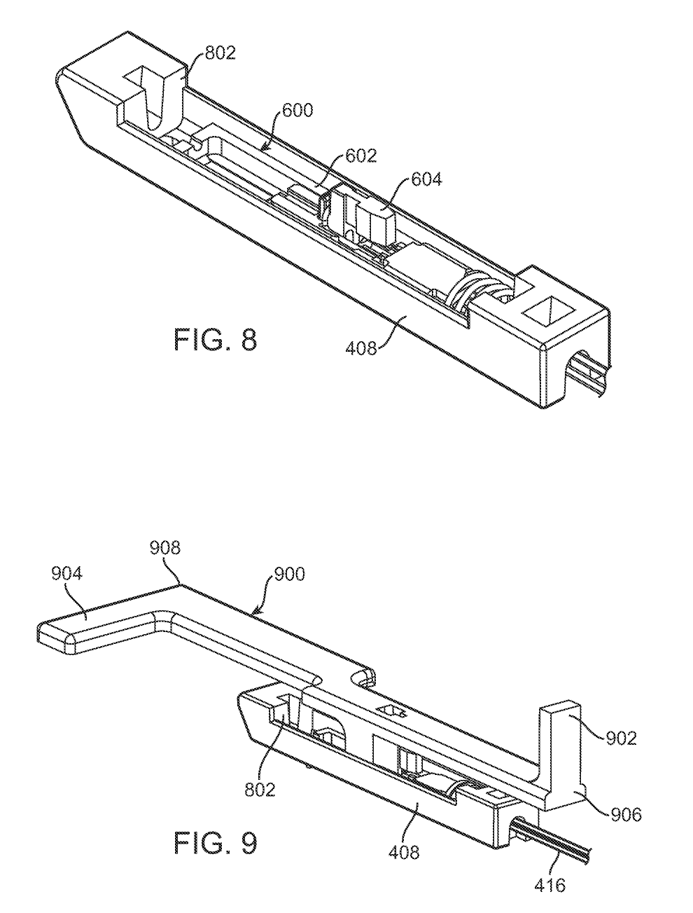

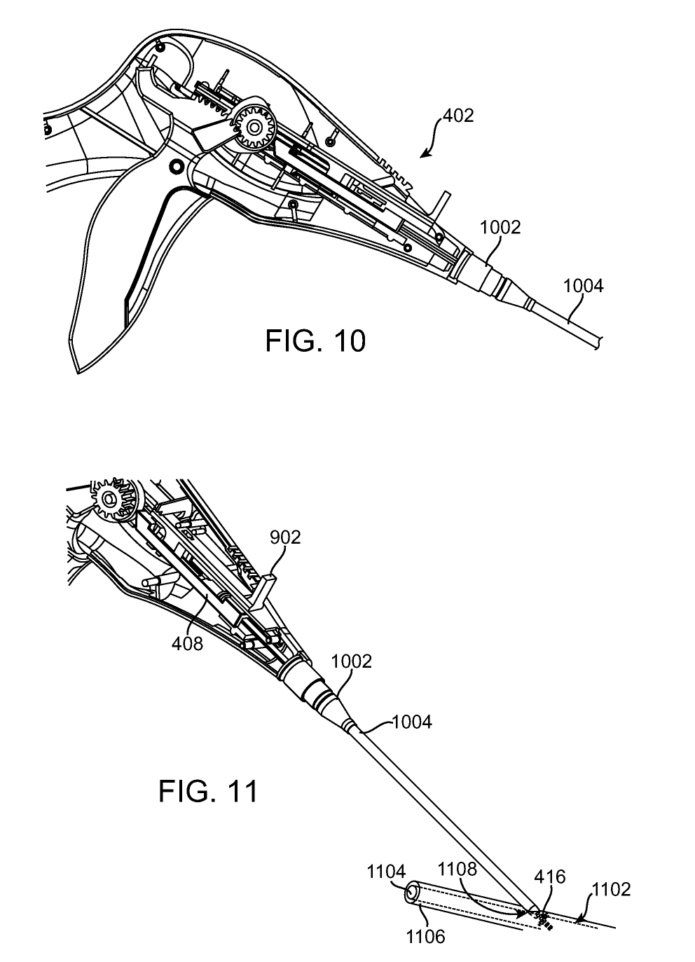

FIG. 8 illustrates retention mechanism 600 nested within front cam follower 408 such that there may be relative linear movement between front cam follower 408 and retention mechanism 600, particularly wire holder component 604. Front cam follower 408 includes a surface 802 facing retention mechanism 600. A slide component 900 is coupled to wire holder component 604 of retention mechanism 600, as shown in FIG. 9, to allow for manual movement of wire holder component 604. Slide component 900 includes a lever 902 extending vertically substantially orthogonal from a distal end 906 of slide component 900. Slide component 900 further includes a rear extension 904 extending horizontally substantially orthogonal from a proximal end 908 of slide component 900. Front cam follower 408, with retention mechanism 600 nested therein and slide component 900 coupled to retention mechanism 600, is disposed within handle 402, as shown in FIG. 4. Lever 902 extends through an opening in the handle housing for access by the clinician, as shown in FIGS. 4 and 10.

FIGS. 10-19 illustrate an embodiment of a method for delivering and implanting an implant such as implant 100 to an arteriotomy in a vessel following an intra-luminal procedure, using the delivery device 400 described with above. Delivery device 400, with implant 100 disposed at a distal end of push rod 404, is inserted into an introducer sheath 1004, as shown in FIG. 10. A coupling device 1002 disposed a proximal end of introducer sheath 1004 snap-fits with a distal end of handle 402. Introducer sheath 1004 may be a procedural sheath used for the intra-luminal procedure such as catheterization, or the procedural sheath may be exchanged for introducer sheath 1004 following the intra-luminal procedure. In either situation, sheath 1004 provides access through a tissue track (not shown) to an arteriotomy 1108 through a wall 1106 in a vessel 1102, as shown in FIG. 11. Sheath 1004 is already disposed in arteriotomy 1108, and push rod 404 and retention feet 416 of delivery device 400 are inserted through sheath 1004 to access lumen 1104 of vessel 1102. Sheath 1004 may be, for example, an 8 French sheath.

Lever 902 is then actuated proximally, as shown in FIG. 11. Actuating lever 902 proximally slides wire holder component 604 proximally, resulting in the position shown in FIG. 7. With wire holder component 604 in the proximal position, retention feet 416 are deployed. Retention feet 416 may be deployed, for example, by including slits in tube 606 and fixing wire 604 to tube 606 distally of the slits. When wire 604 is drawn proximally, tube 606 compresses and buckles at the slits, as described, for example, in U.S. Pat. No. 6,767,356 to Kanner et al., the entirety of which is incorporated by reference herein. Other methods and devices to deploy retention feet may be utilized, as would be apparent to one of ordinary skill in the art.

Upon deployment of retention feet 416, the clinician manually retracts the entire assembly (i.e., handle 402, push rod 404, retention feet 416, implant 100, and sheath 1004) until tactile feedback is felt, signifying retraction of introducer 1004 from vessel 1102, as well as contact of deployed retention feet 416 with an inside surface of vessel wall 1106.

Trigger 412 of handle 402 is then actuated to deliver implant 100 and to advance and undeploy retention feet 416 for removal. In particular, trigger 412 is pulled proximally, resulting in counter-clockwise rotation of cam gear 406. At a prescribed stage of trigger 412 rotation, for example, 25 degrees, as shown in FIG. 13, cam gear 406 rotates such that a surface thereof contacts front cam follower 408. Forward (i.e. distal) translation of front cam follower 408 causes implant 100 to be moved distally. Further, front cam follower 408 moves relative to retention mechanism 600 until surface 802 of front cam follower 408 contacts retention mechanism 600, thereby initiating distal advancement of retention mechanism 600.

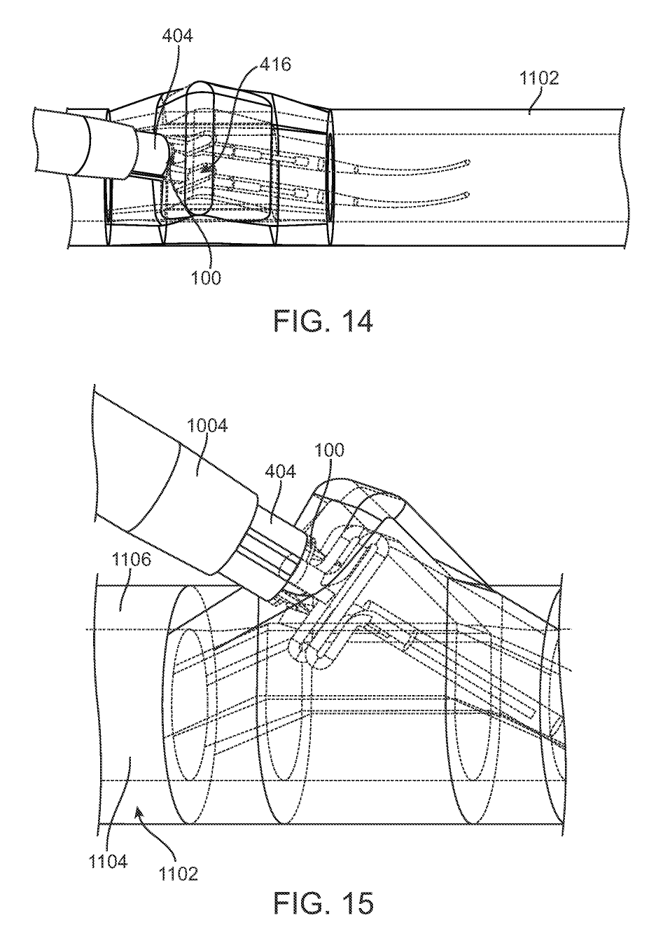

Continued rotation of trigger 412 causes continued translation of implant 100 so that tapered plug 104 is fully pushed into the arteriotomy 1108 and arteriotomy 1108 conforms to the oblong shaped proximal end 118 of tapered plug 104. Further, prongs 108 pierce the tissue of the vessel wall 1106 and barbs 114 are submerged in the tissue such that implant 100 is securely anchored in place. At this point, distally facing surface 112 of head 102 is in intimate contact with vessel wall 1106, as shown in FIGS. 14 and 15.

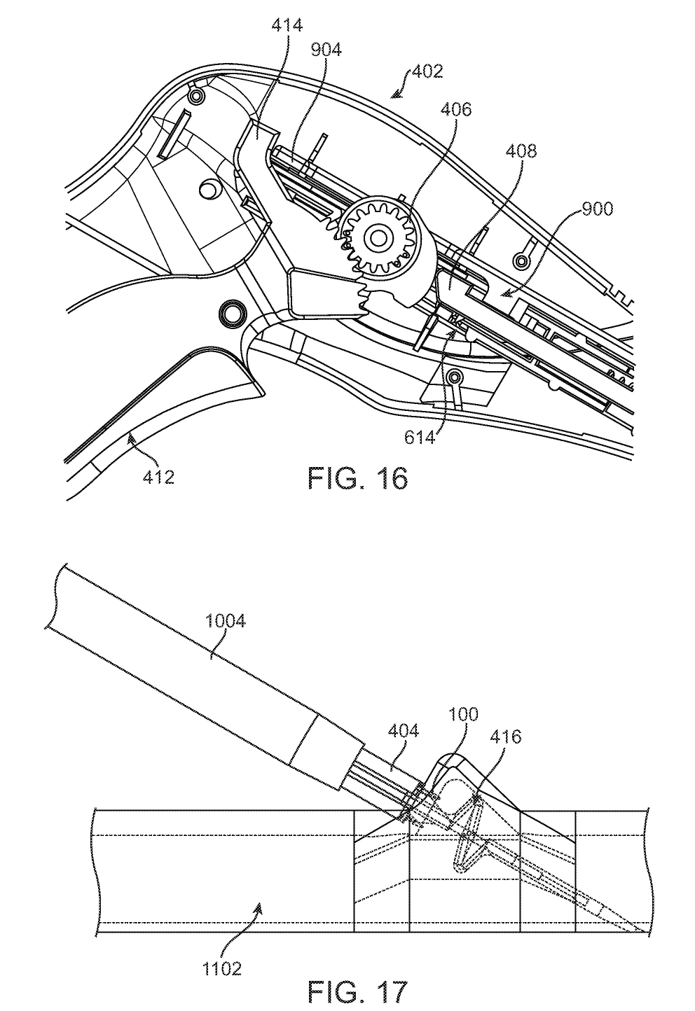

Referring to FIG. 16, after implant 100 is anchored in place, at another prescribed stage of rotation of trigger 412 (e.g. approximately 30 degrees), extension 614 of tube holder component 602 abuts an internal surface in handle 402. Such contact stops tube holder component from moving distally. Further, cam gear 406 begins to clear forward cam follower 408 and rear arm projection 414 of trigger 412 abuts rear extension 904 of slide component 900. As trigger 412 continues to be rotated, tube holder component 602 stops translating distally while rear arm projection 414 continues to translate wire holder component 604 distally. Such motion causes retention mechanism 600 to move from the position shown in FIG. 7 with wire holder 604 in the proximal position corresponding to retention feet 416 in the deployed position, to the position shown in FIG. 6 with wire holder component 604 in the distal position corresponding to the retention feet 416 in the undeployed position. FIG. 17 illustrates retention feet 416 as they start to undeploy and FIG. 18 shows retention feet 416 in the undeployed, straightened position.

After implant 100 is secured in place and retention feet 416 are in the undeployed, straightened position, delivery device 400 is withdrawn proximally such that retention feet 416 exit vessel 1102 through slots 110 in implant 100, leaving implant 100 in place in vessel 1103, as shown in FIG. 19.

While various embodiments of the present disclosure have been described above, it should be understood that they have been presented by way of illustration and example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. For example, and not by way of limitation, the implant described in FIGS. 1-3 can be implanted using a delivery device and method other than the delivery device and method described with respect to FIGS. 4-19. Similarly, the delivery device and method described with respect to FIGS. 5-19 can be used to deliver an implant different than the one described with respect to FIGS. 1-3. All patents and publications discussed herein are incorporated by reference herein in their entirety.

What is claimed is:

1. A device for sealing a passage through a vessel wall comprising:
a head having a proximal surface and a distal surface;
a tapered plug having a distal end and a proximal end adjoining the head distal surface; and
a plurality of prongs extending from the distal surface,
wherein the head is configured such that the distal surface faces an outside surface of the vessel wall, the tapered plug is configured to be placed in the passage, and the prongs are configured to enter tissue of the vessel wall surrounding the passage.

2. The device of claim 1, wherein the prongs each include a barb.

3. The device of claim 1, wherein the plug proximal end is generally oblong in shape and the plug distal end is generally circular in shape.

4. The device of claim 3, wherein the head is generally circular in shape and a diameter of the head is larger than a long axis of the plug proximal end.

5. The device of claim 1, wherein the head is generally circular in shape and a diameter of the head is larger than the plug proximal end.

6. The device of claim 1, wherein the prongs are distributed around a periphery of the head.

7. The device of claim 6, wherein the prongs are equally distributed around the periphery of the head and extend generally orthogonally from the head distal surface.

8. The device of claim 1, wherein the device is bioabsorbable.

9. The device of claim 8, wherein the device is made of a material selected from the group consisting of stainless steel 316L, stainless steel 316 LVM, titanium, magnesium, polymethylmethacrylate (PMMA), high density polyethylene (HDPE), ultra high molecular weight polyethylene (UHMWPE) polyglycolic acid (PGA), polylactic acid (PLA), alloys or blends of PGA and PLA, alloys or blends of PGA and tri-methyl carbonate, and alloys or blends of PLA and tri-methyl carbonate.

10. The device of claim 1, wherein the head further includes a pair of slots extending there through.

11. A method for closing a puncture in a wall of a vessel, comprising the steps of:
providing an implant having a head with a proximal surface and a distal surface, a tapered plug having a proximal end adjoining the distal surface, and a plurality of prongs extending from the distal surface;
delivering the implant to the puncture;
implanting the implant such that the distal surface of the head faces an outside surface of the vessel wall, the tapered plug is placed in the puncture, and the prongs are inserted into tissue of the vessel wall surrounding the puncture.

12. The method of claim 11, wherein the prongs each include a barb.

13. The method of claim 11, wherein the plug proximal end is generally oblong in shape and a distal end of the plug is generally circular in shape.

14. The method of claim 13, wherein the head is generally circular in shape and a diameter of the head is larger than a long axis of the plug proximal end.

15. The method of claim 11, wherein head is generally circular in shape and a diameter of the head is larger than the plug proximal end.

16. The method of claim 11, wherein the prongs are distributed around a periphery of the head.

17. The method of claim 16, wherein the prongs are equally distributed around the periphery of the head and extend generally orthogonally from the head distal surface.

18. The method of claim 11, wherein the device is bioabsorbable.

19. The method of claim 18, wherein the device is made of a material selected from the group consisting of stainless steel 316L, stainless steel 316 LVM, titanium, magnesium, polymethylmethacrylate (PMMA), high density polyethylene (HDPE), ultra high molecular weight polyethylene (UHMWPE) polyglycolic acid (PGA), polylactic acid (PLA), alloys or blends of PGA and PLA, alloys or blends of PGA and tri-methyl carbonate, and alloys or blends of PLA and tri-methyl carbonate.

20. The method of claim 11, wherein the step of delivering the implant to the puncture includes delivering a pair of retention wires to a lumen of the vessel, wherein the head of the implant includes a pair of slots disposed there through, and wherein, after the step of implanting the implant, the wires are removed from the vessel through the slots.
